(19) United States
(12) Patent Application Publication
(10) Pub. No.: US 2010/0076422 A1
(43) Pub. Date: Mar. 25, 2010

(54) Thermal Treatment of Nucleus Pulposus

(75) Inventor: Ronald J. Podhajsky, Boulder, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/236,686

(22) Filed: Sep. 24, 2008

(51) Int. Cl. A61B 18/04; A61B 19/00

(52) U.S. Cl. 606/32; 128/898; 606/33; 606/27

(57) ABSTRACT

A method for relieving pain associated with an intervertebral disc having a disc nucleus pulposus is provided. The method includes the initial step of: providing an elongated probe member having proximal and distal ends and defining a longitudinal axis, and having a flexible guidable region adjacent the distal end. The method also includes the steps of: introducing the flexible guidable region of the probe into the nucleus pulposus of the intervertebral disc and supplying energy to the guidable region from an energy source, to heat or induce an electromagnetic field within the nucleus pulposus sufficient to denature proteins expressing at least one inflammatory cytokine.

### BACKGROUND

1. Technical Field

The present disclosure relates to methods for treating intervertebral disc problems using percutaneous techniques without the need for major surgical intervention, and more particularly, to methods for the insertion of a cannula into the intervertebral disc and the insertion of a thermal probe into the disc material to heat the intervertebral disc thereby relieving and treating abnormalities or pain related to the disc.

2. Background of Related Art

The use of thermal therapy in and around the spinal column is known. Also, the insertion of cannula into the intervertebral discs is commonly done for injection of contrast mediums to implement X-ray discograms. This technique is used to detect or diagnose abnormalities or damage to the intervertebral disc. The use of heating of an intervertebral disc to relieve pain is described in U.S. Pat. No. 5,433,739, issued Jul. 18, 1995, and in U.S. Pat. No. 5,571,147, issued Nov. 5, 1996, the entire contents of each of which being incorporated herein by reference. In these patents, electrodes are described for either radiofrequency or resistive thermal heating of all or a portion of the intervertebral disc. Straight, curved, and flexible-tipped electrodes are described for this purpose. The thermal treatment of an intervertebral disc for the relief of back pain is also described within the patents cited above.

The use of a resistively heated probe adapted to be inserted into the intervertebral disc is described in U.S. Pat. No. 6,073,051, issued Jun. 6, 2000, the entire content of which is incorporated herein by reference. The U.S. Pat. No. 6,073,051 discloses an apparatus or probe for treating intervertebral discs, the apparatus including a flexible catheter which is introduced into the nucleus pulposus and manipulated into an inner wall of the annulus fibrosus along annulus fibrosus/nucleus pulposus interface. Accordingly, functional element or intradiscal section of catheter delivers a therapeutic effect to the area of nucleus pulposus to be treated, i.e., fissures.

It is desirable to treat the posterior or posterior/lateral portion of the intervertebral disc for the indication of mechanical degeneration of the disc and discogenic back pain. Pain can be derived from degeneration or compression of the intervertebral disc in its posterior or posterior/lateral portions. There is some innervation of the intervertebral disc near the surface of the disc and also within the outer portion known as the annulus fibrosus. Fissures or cracks within the disc caused by age, mechanical trauma, or disc degeneration are believed to be associated with painful symptoms.

### SUMMARY

According to one aspect of the present disclosure a method for relieving pain associated with an intervertebral disc having a disc nucleus pulposus is provided. The method includes the initial step of: providing an elongated probe member having proximal and distal ends and defining a longitudinal axis therethrough, the probe having a flexible guidable region adjacent the distal end. The method also includes the steps of: introducing the flexible guidable region of the probe into the nucleus pulposus of the intervertebral disc and supplying energy to the guidable region from an energy source, to heat or induce an electromagnetic field within the nucleus pulposus sufficient to denature proteins expressing at least one inflammatory cytokine.

A method for relieving pain associated with an intervertebral disc having a nucleus pulposus is also contemplated by the present disclosure. The method includes the steps of: introducing at least one of a thermal and electromagnetic transmitting element of a probe into the nucleus pulposus and supplying at least one of thermal and electromagnetic energy from an energy source to at least one of the thermal and electromagnetic transmitting element to denature proteins expressing tumor necrosis factor-alpha.

### BRIEF DESCRIPTION OF THE DRAWINGS

The features of the apparatus and method of the present disclosure will become more readily apparent and may be better understood by referring to the following detailed description of illustrative embodiments of the present disclosure, taken in conjunction with the accompanying drawings, wherein:

FIG. 1 is a side view of a portion of the spine;

FIG. 2 is an enlarged side view of the area indicated as “2” of the spine of FIG. 1;

FIG. 3 is a cross-sectional plan view of a cervical disc and vertebra;

FIG. 4 is a cross-sectional view of an intervertebral disc;

FIG. 5 is a schematic illustration of an intervertebral apparatus, in a disassembled condition, depicting an insertion cannula, a thermal or EMF probe and associated auxiliary electronic components; and

FIG. 6 is a cross-sectional plan view of an intervertebral disc with a portion of an intervertebral apparatus inserted therein according to yet another method or another step of the present disclosure.

### DETAILED DESCRIPTION

The present disclosure provides for a method for the treatment of intervertebral discs. In particular, according to one aspect of the present disclosure, a method for relieving pain associated with an intervertebral disc having a disc nucleus pulposus and an outer annulus fibrosus surrounding the nucleus pulposus, is provided. Such disorders include but are not limited to degenerative discs with (i) localized tears or fissures in the annulus fibrosus, (ii) localized disc herniations with contained extrusions, and (iii) chronic, circumferential bulges.

It will be readily apparent to a person skilled in the art that the apparatus and method of use of the apparatus may be used to treat/destroy body tissue in any body cavity or tissue locations that are accessible by percutaneous or endoscopic catheters or open surgical techniques, and is not limited to the disc area. Application of the apparatus and method in all of these organs and tissues are intended to be included within the scope of the present disclosure.

In the drawings and in the following description, the term “proximal”, as is traditional, will refer to the end of the apparatus, or component thereof which is closest to the operator, and the term “distal” will refer to the end of the apparatus, or component thereof, which is more remote or further from the operator.

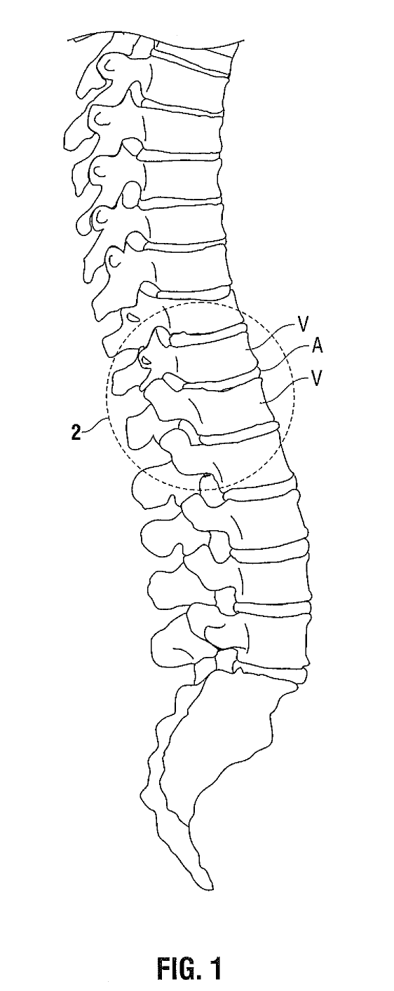

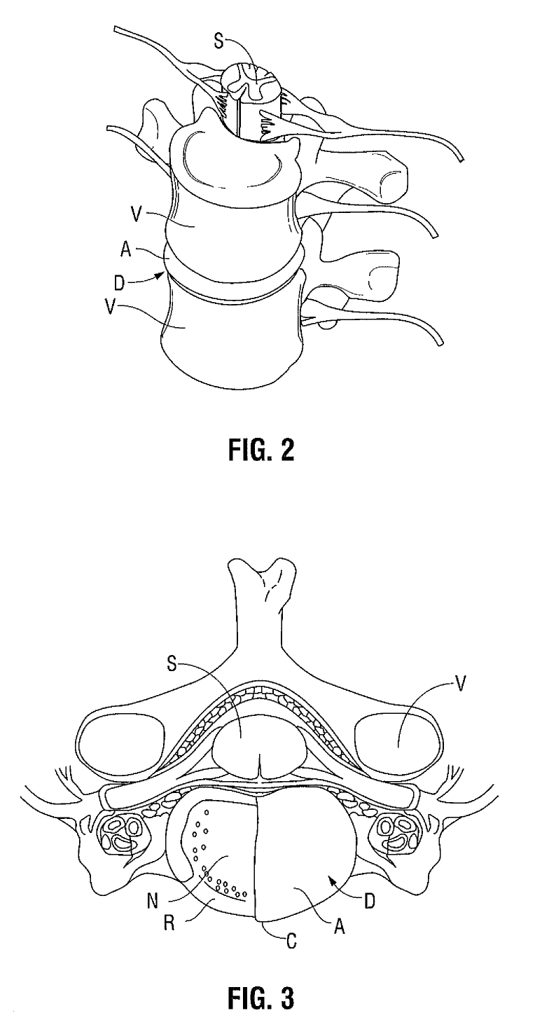

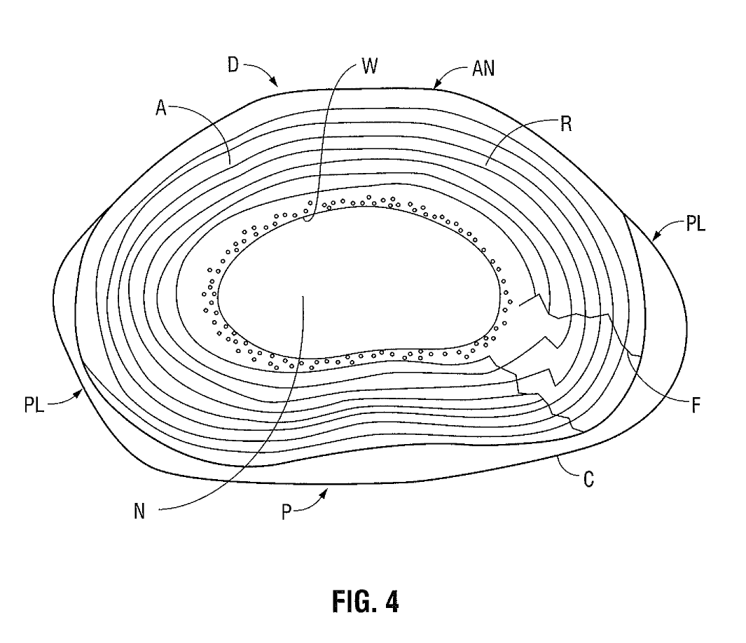

Prior to a detailed discussion of the apparatus and method according to the present disclosure, a brief overview of the anatomy of the intervertebral disc and surrounding anatomical structures are presented. Accordingly, as seen in FIGS. 1-4, a spinal column is shown having a plurality of vertebrae “V” with intervertebral discs “D” disposed therebetween. With reference to FIGS. 2 and 3, the vertebrae “V” include a canal, vertebral foramina, for the protection of the medulla spinalis (spinal cord “S”).

As shown in FIGS. 2-4, the intervertebral disc “D” includes a nucleus pulposus “N” disposed within annulus fibrosus “A”. Annulus fibrosus “A” includes a tough fibrous material that defines a plurality of annular cartilaginous rings “R” forming the natural striata of annulus fibrosus “A”. Nucleus pulposus “N” is made up primarily of an amorphous gel having a softer consistency than annulus fibrosus “A”. Nucleus pulposus “N” usually contains 70%-90% water by weight and mechanically functions similar to an incompressible hydrostatic material. The juncture or transition area of annulus fibrosus “A” and nucleus pulposus “N” generally defines, for discussion purposes, an inner wall “W” of annulus fibrosus “A”. Disc cortex “C” surrounds annulus fibrosus “A”. Posterior, anterior, and lateral aspects of intervertebral disc “D” are identified as “P”, “AN” and “L”, respectively, with the opposed posterior-lateral aspects identified as “PL”. In FIG. 2, a portion of intervertebral disc “D” has been cut away so that half of the vertebral body may be more easily visualized.

When mechanical stress is put upon a disc or when a disc degenerates with age, fissures, illustrated by cracks “F” in FIG. 4, may occur in the posterior or posterior/lateral portions of disc “D”. Problems with nerves, fissures “F” and degenerative discs may give rise to various patient problems, such as back or leg pain originating from the irritation or occurrence of these abnormalities. Moreover, these conditions may ultimately result in conditions such as bulging or herniated discs.

One possible mechanism for the pain associated with damaged or herniated discs, involves various pathophysiological agents, such as tumor necrosis factor-alpha (TNFα), expressed in vivo by the herniated nucleus pulposus “N.” As was demonstrated experimentally, application of nucleus pulposus “N” extracted from a herniated disc induces morphologic and functional changes in the nerve root and results in pain-related behavior. It was also shown that TNFα also produces neuropathologic changes to the nerve root mimicking the changes effected by the nucleus pulposus “N.” The results of the study are reported in a publication Tainaki Igarashi et al., Exogenous Tumor Necrosis Factor-Alpha Mimics Nucleus Pulposus-Induced Neuropathology, SPINE, Vol. 25, No. 23, pp. 2975-2980 (2000), which is incorporated by reference in its entirety herein. It is also believed that additional cytokine constituents of nucleus pulposus “N” may be responsible for neuropahological changes associated with herniated discs “D.” Therefore, it is believed that TNFα is a key pathogenic factor in producing various neuropathic pain states associated with herniated discs.

The herniated disc “D” expresses a number of cytokines, such as TNFα, from the nucleus pulposus “N” through the fissures “F” in the annular cartilaginous rings “R.” The expressed cytokines then permeate the spinal cord “S” inflaming the nerves therein. The diffusion rate of TNFα is based on TNFα diffusion through a tight and highly viscous net of glycosaminoglycans and branching structural proteins of the extracellular matrix, that serve as a reservoir of cytokines and growth factors. Therefore, the amount of TNFα expected to be effective in causing nerve injury is expected to be lower at the nerve root barrier than at the core of the disc “D.”

Once TNFα contacts the nerve fibers within the spinal column “S” and nerve injury occurs, the TNFα protein expression is upregulated. Interference with TNFα upregulation may reduce magnitude of the nerve injury, thereby reducing the duration of the pain state. This may be achieved by applying thermal, cryogenic or electromagnetic field (EMF) therapy on intervertebral disc “D”, in particular to the nucleus pulposus “N.” It is believed that this results in denaturations of proteins responsible for the upregulation of TNFα, which, in turn, decreases supply of TNFα to the nerve fibers of the spinal cord “S” thereby relieving painful states associated with TNFα. Thus, it is desirable to have a practical and efficient method of placing a treatment probe into the nucleus pulposus “N” of disc “D” where TNFα is produced and expressed.

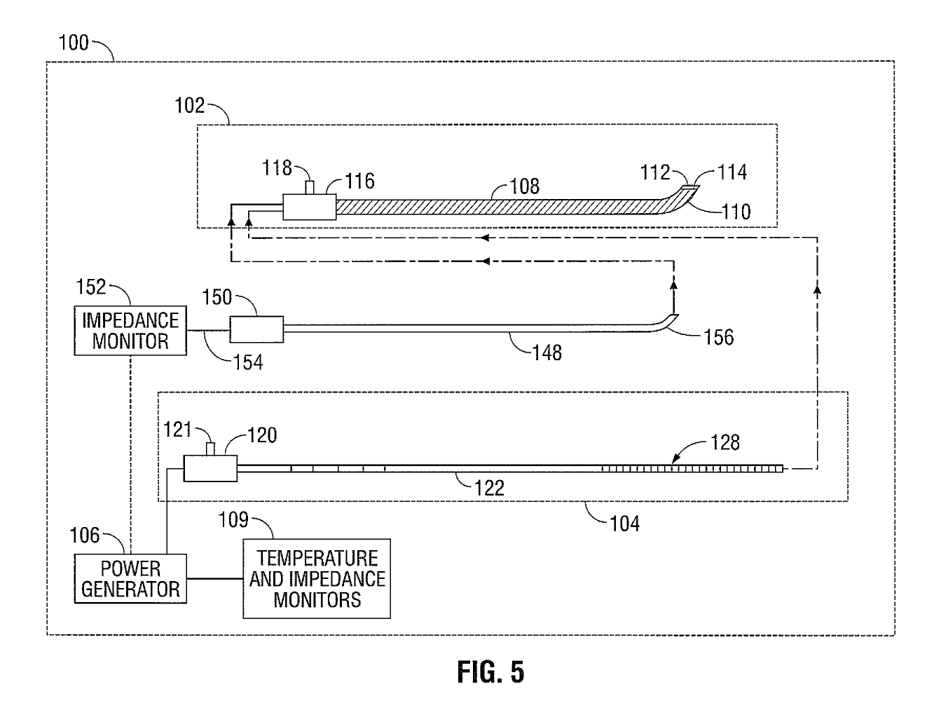

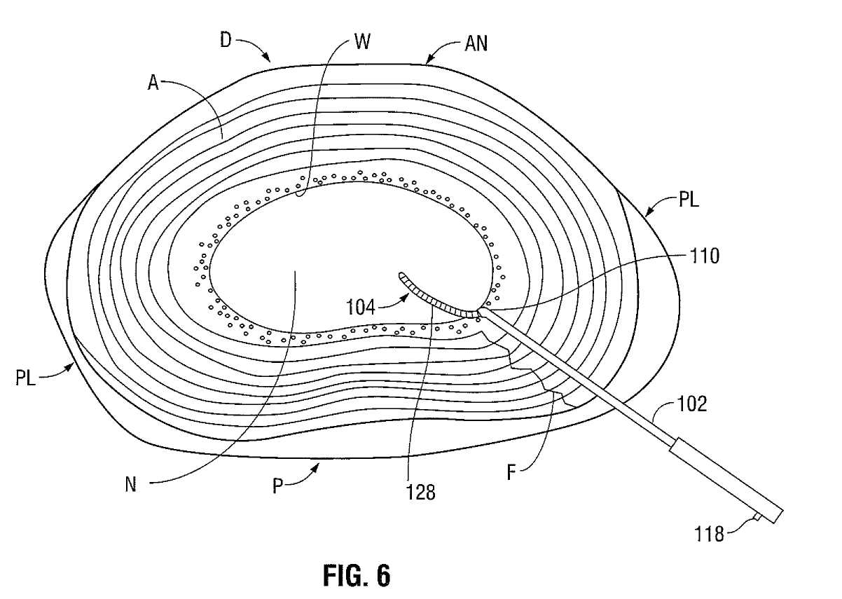

With reference to FIG. 5, an apparatus according to the present disclosure is shown and is generally designated as apparatus 100. Apparatus 100 includes an outer insertion or introducer cannula 102 and a probe 104 adapted to deliver thermal, cryogenic, microwave or EMF energy. The probe 104 is positionable within cannula 102, and a power source 106 or supply of cryogenic fluid or gas, is connected to the probe 104. The thermal probe 104 includes a shaft 122 having a guidable region 128, which may be pre-bent to obtain desirable orientation of the distal tip of the probe.

Introducer cannula 102 includes a rigid tubular shaft 108 defining a longitudinal axis “X” and having a rigid curved or arcuate portion 110 adjacent a distal end thereof angularly offset with respect to the longitudinal “X” axis at an angle ranging from about 15° to about 45°, or in particular embodiments, about 23°. Shaft 108 includes of a conductive material such as stainless steel and is insulated with insulation along most of the length thereof as indicated by the hatching of FIG. 5. Alternatively, shaft 108 may be fabricated from an insulative material, such as suitable polymeric materials formed by conventional injection molding techniques. The distal end portion 112 of shaft 108 may be left uninsulated or exposed to permit electrical connection to or contact with the tissue as cannula 102 is placed in the tissue (e.g., for impedance measuring, etc.). Alternatively, exposed portion 112 may be connected to power source 106 to heat, stimulate or generate micro-thermal energy within the tissue to facilitate passage through the tissue.

A distal tip 114 of shaft 108 may be sharpened to facilitate penetration into the disc tissue, e.g., through the bone of the cortex “C” and annulus fibrosus “A” into nucleus pulposus “N.” A handle or housing 116 is connected to the proximal end of cannula shaft 108 to facilitate manipulation of cannula 102. Handle 116 includes an index marker 118 to indicate the direction of arcuate portion 110 of cannula 102 such that when the probe 104 is introduced within cannula 102, the surgeon may determine in which azimuthal rotational direction the curve is oriented.

Cannula shaft 108 may have a diameter ranging from a fraction of a millimeter to several millimeters and a length of a few centimeters up to about 20 centimeters or more. Alternatively, cannula shaft 108 may be fabricated from an MRI compatible material, including cobalt alloys, titanium, copper, nitinol, etc. Arcuate portion 110 of cannula 102 may assume a variety of angular orientations depending on the surgical procedure to bee performed. In an embodiment for thermal or EMF therapy of the intervertebral disc, arcuate portion 110 is arranged such that the probe 104 is generally delivered from cannula 102 in a substantially orthogonal relation to the longitudinal “X” axis.

Power source or generator 106 may be, for example, a radiofrequency generator providing energy at frequencies between several kilohertz to several hundred megahertz. Power source 106 may have a power output ranging from several watts to several hundred watts, depending on clinical need. Power source 106 may have control devices to increase or modulate power output as well as readout and display devices to monitor energy parameters such as voltage, current, power, frequency, temperature impedance 109, etc., as appreciated by one skilled in the art. Other types of power sources are also contemplated, e.g., including resistive heating units, laser sources, or microwave generators.

Apparatus 100 may include an imaging system (not shown) for potentially monitoring, controlling or verifying the positioning of cannula 102 and/or thermal probe 104. Imaging systems that are contemplated include X-ray machines, fluoroscopic machines or an ultrasonic, CT, MRI, PET, or other imaging devices. Several of these devices have conjugate elements (not shown), on the opposite side of the patient's body, to provide imaging data. For example, if the imaging system is an X-ray machine, the conjugate element may be a detection device, such as an X-ray film, digital X-ray detector, fluoroscopic device, etc. Use of imaging machines to monitor percutaneously placed electrodes into tissue is commonly practiced in the surgical field.

With continued reference to FIG. 5, apparatus 100 further includes a stylet 148 which may be used in conjunction with cannula 102. Stylet 148 is positionable within the lumen of cannula 102 and occludes the front opening of cannula 102 to prevent entry of tissue, fluids, etc., during introduction of cannula 102 within intervertebral disc “D”. Stylet 148 includes a proximally positioned hub 150 which mates with handle 116 of cannula 102 to lock the components together during insertion.

Stylet 148 can be made from a rigid metal tubing with either a permanent bend 156 at the distal end to correspond to the curvature of arcuate portion 112 of cannula 102 or may be a straight guide wire that adapts to the curvature of cannula 102 when the guide wire is inserted within cannula 102. Hubs 116, 120, 150 and connector 154 can take various forms including luer hubs, plug-in-jack-type connections, integral cables, etc.

An impedance monitor 152 is also be provided that is connected, as shown by connection 154, to stylet 148. The impedance monitor 152 communicates electrically with the exposed portion 112 of cannula 102. Stylet 148 is introduced into cannula 102 to monitor impedance of the tissue adjacent the distal end of cannula 102. Alternatively, connection of the impedance monitor 152 may be made directly to the shaft of cannula 102 whereby impedance measurements are effectuated through the exposed distal end of cannula 102. Once the combination of stylet 148 and cannula 102 are inserted into the body, impedance monitoring assists in determining the position of cannula tip 112 with respect to the patient's skin, cortex “C” of disc “D”, annulus fibrosus “A”, and/or nucleus pulposus “N” of disc “D,” since these regions have easily identifiable different impedance levels.

For a fully insulated electrode or cannula with an exposed area of a few square millimeters at the cannula tip 112, the impedance changes as the cannula tip 112 is transitioned from the cortex “C” of disc “D” into annulus fibrosus “A” and eventually into the nucleus “N” of disc “D”. Differences of impedance may range from a few hundred ohms outside the disc “D”, to 200 to 300 ohms in annulus fibrosus “A”, to approximately 100 to 200 ohms in nucleus “N”. This variation may be detected by the surgeon by visualizing impedance on meters or by hearing an audio tone which is proportional to impedance generated by monitor 109. Thus, detecting changes in impedance allows for detection and proper placement of the curved cannula within disc “D”. This also allows for precise placement of the probe 104 within the nucleus pulposus “N.”

Use of apparatus 100 for thermal treatment of an intervertebral disc is discussed with respect to FIGS. 5 and 6. With reference to FIG. 6, the targeted intervertebral disc “D” is identified during a pre-operative phase of the surgery. Access to the intervertebral disc area is then ascertained through percutaneous techniques or open surgical techniques.

Cannula 102, with stylet 148 positioned and secured therein, is introduced within intervertebral disc “D” near a location that is in relative close proximity to or adjacent to the region of intervertebral disc “D” to be thermally or electromagnetically treated, such as the within the nucleus pulposus “N.” Cannula 102 may also be utilized without stylet 148 depending on a particular surgical procedure.

Impedance monitoring is utilized to determine the position of cannula tip 114 with respect to the patient's skin, cortex “C” of disc “D”, annulus fibrosus “A” and/or nucleus “N” of disc “D”. As discussed above, these regions have different and quantifiable impedance levels thereby providing an indication to the user of the position of cannula tip 114 within the tissue. Monitoring of the location of cannula 102 may also be confirmed with an imaging system (not shown).

Stylet 148 is then removed from cannula 102 and the probe 104 is positioned within the internal lumen of cannula 102 and advanced through cannula 102. The pre-bent orientation of guidable region 128 is arranged to coincide with the arcuate end portion 110 of cannula 102. Confirmation of this orientation may be made with the location of the indexing element 121 of handle 120 (see FIG. 5). The arcuate end position 110 is articulated to directly access the posterior-lateral “PL” section of annulus fibrosus “A” allowing the end portion 110 to enter nucleus “N”. The probe 104 is thereafter advanced to position guidable region 128 medially through the posterior “P” section of annulus fibrosus “A” and into the nucleus pulposus “N” as seen in FIG. 6. Guidable region 128 of probe 104 is extended by about 1.5 cm from the distal end of cannula 102 into the nucleus pulposus “N.”

As seen in FIG. 6, cannula 102 may be positioned so as to place arcuate end portion 110 of cannula 102 in the desired location and orientation within annulus fibrosus “A”. The arcuate end portion 110 is positioned in close proximity to inner wall “W” of annulus fibrosus “A”. When so positioned, as will be described in greater detail below, advancement of thermal probe 104 through cannula 102 results in placement of guidable region 128 in the nucleus “N” of the intervertebral disc “D.”

Following the confirmation that guidable region 128 of probe 104 is properly placed, “Simulation Mode” is selected on power source 106. First, the “Sensory Range” is activated and the amplitude of the simulation is increased until indications of effect and/or stimulation, of the region to be treated, are obtained. The amplitude at which the indications of effect and/or stimulations are obtained, of the region to be treated, is then noted. In the event that the “Sensory Range” does not provide a sufficient effect, the “Motor Range” is activated and the amplitude is increased. The noted amplitude dictates the temperature that is selected on the “Automatic Temperature Control” for the treatment of disc “D”. Accordingly, the heating cycle for each position of guidable region 128 of probe 104 is dictated by the threshold of the stimulations,

In one embodiment, if stimulation of the region to be treated occurs below about 0.75V, then a temperature of approximately 60° C. is applied. In another embodiment, if stimulation of the region to be treated occurs between about 0.75V and 1.25V, then a temperature of approximately 65° C. is applied. In a further embodiment, if stimulation of the region to be treated occurs above about 1.25V, then a temperature of approximately 70° C. is applied. A temperature approximately equal to the boiling point of the nucleus “N” and up to approximately 90° C. is applied if stimulation occurs above about 1.5V when the guidable region 128 of thermal probe 104 is placed within nucleus “N.” Heat treatment of the nucleus pulposus “N” denatures inflammatory proteins in the nucleus pulposus “N” which are responsible for expressing TNFα and other cytokines associated with inflammatory response. This, in turn, relieves the pain associated with the herniated disc “D.” [please provide specific temperature ranges associated with TNF protein disassociation as well as other treatment methods, probe placement etc.]

Once guidable region 128 of probe 104 is positioned within nucleus pulposus “N” as desired, power source 106 is activated whereby the probe 104 delivers thermal energy and/or creates an electromagnetic field through guidable region 128 therein. Appropriate amounts of power, current or thermal heat may be monitored from the external power source 106 and delivered for a certain amount of time as determined appropriate for clinical needs.

As appreciated, the degree of extension of guidable region 128 from cannula 102 controls the volume of disc tissue heated by probe 104. A thermal sensor (not shown), provided on the probe 104 can provide information concerning the temperature of tissue adjacent the distal end. In an embodiment, impedance measurements of the tissue provide an indication of the degree of desiccation, power rise, or charring, that may be taking place near tip 134 of thermal probe 104. This indicates the effectiveness of the treatment and guards against unsafe contraindications of the therapy.

The site of injury and/or the region to be treated receives a higher level of directed RF energy by extending the guidable region 128 into the tissue. As a result, the likelihood of effective treatment of the site of injury and/or the region to be treated is increased. The increased effective treatment may also include directed RF energy denaturing of the biochemical constituents of the nucleus pulposus to thereby reduce their contribution as a source of pain. Additionally, the directed RF energy may also create a local area of reduced pressure and higher viscosity in the nucleus “N”, in the immediate vicinity of the fissure(s) to thereby reduce the likelihood of further extravasations of nuclear material.

One advantage of the present apparatus 100 and method is that by using a curved introduction cannula, effectiveness of the probe in difficult lumbar or lumbar-sacral intervertebral discs is increased. In these approaches, nearby heavy bone structure, such as the iliac crest, can often obscure a placement of a curved probe parallel to the end plates or bony margins of adjacent intervertebral discs. By appropriate articulation and rotation of a curved cannula, the extension of the probe, parallel to the so-called end plates of the intervertebral discs, is made possible with minimal repositioning and manipulation of the introduction cannula.

A further advantage of the present apparatus 100 and method is that the apparatus 100 enables simple, minimally-invasive, percutaneous, out-patient treatment of intradiscal pain without the need for open surgery necessary for discectomies or spinal stabilization using plates, screws, and other instrumentation hardware. A further advantage of the present disclosure is that the apparatus 100 is simple to use and relatively economical. Compared to open disc surgery, the treatment of the disc by percutaneous electrode placement requires less surgical time a few hours with minimal hospitalization, and with minimal morbitity to the patient. On the other hand, open surgical procedures often require full anesthesia, extensive operating room time, and longer hospital and home convalescence.

While the above description contains many specific examples, these specifies should not be construed as limitations on the scope of the disclosure, but merely as exemplifications of embodiments thereof. Those skilled in the art will envision many other possible variations that are within the scope and spirit of the disclosure as defined by the claims appended hereto.

What is claimed is:

1. A method for relieving pain associated with an intervertebral disc having a disc nucleus pulposus, the method comprising the steps of:
providing an elongated probe member having proximal and distal ends and defining a longitudinal axis therethrough, the probe having a flexible guidable region adjacent the distal end;
introducing the flexible guidable region of the probe into the nucleus pulposus of the intervertebral disc; and
supplying energy to the guidable region from an energy source, to at least one of heat and induce an electromagnetic field within the nucleus pulposus sufficient to denature proteins expressing at least one inflammatory cytokine.

2. The method according to claim 1, further including the steps of:
positioning a cannula adjacent a region of the intervertebral disc to be treated; and
passing the flexible guidable region of the probe through a lumen defined in the cannula.

3. The method according to claim 2, wherein the cannula includes an arcuate portion adjacent a distal end thereof and wherein, during the step of introducing the flexible guidable region of the probe, the arcuate cannula portion guides the flexible guidable region of the probe adjacent to the region to be treated.

4. The method according to claim 3, further comprising the step of:
articulating the arcuate portion of the cannula to a desired orientation within the intervertebral disc.

5. The method according to claim 4, further comprising the step of:
monitoring impedance of tissue to detect variations in tissue-type to thereby facilitate positioning of the flexible guidable region of the probe.

6. The method according to claim 5, further comprising the steps of:
increasing an amplitude of at least one of thermal and electromagnetic energy supplied to the probe until indications of effect on the intervertebral disc are obtained; and
noting the amplitude at which the indications of effect of the intervertebral disc are obtained.

7. The method according to claim 6, wherein when the indications of effect of the intervertebral disc are obtained for amplitudes below about 0.75 volts, the method includes the step of applying thermal energy at about 60° C.

8. The method according to claim 6, wherein when the indications of effect of the intervertebral disc are obtained for amplitudes between about 0.75 volts and 1.25 volts, the method includes the step of applying thermal energy at about 65° C.

9. The method according to claim 6, wherein when the indications of effect of the intervertebral disc are obtained for amplitudes above about 1.25 volts, the method includes the step of applying thermal energy at about 70° C.

10. The method according to claim 6, wherein when the indications of effect of the intervertebral disc are obtained for amplitudes above about 1.5 volts, the method includes the step of applying thermal energy at about 90° C.

11. The method according to claim 6, wherein the at least one inflammatory cytokine is tumor necrosis factor alpha.

12. A method for relieving pain associated with an intervertebral disc having a nucleus pulposus, the method comprising the steps of:
introducing at least one of a thermal and electromagnetic transmitting element of a probe into the nucleus pulposus; and
supplying at least one of thermal and electromagnetic energy from an energy source to at least one of the thermal and electromagnetic transmitting element to denature proteins expressing at least one inflammatory cytokine.

13. The method according to claim 12, further comprising the step of:
heating the nucleus pulposus to about 90° C.

14. The method according to claim 12, wherein the at least one inflammatory cytokine is tumor necrosis factor-alpha.

15. The method according to claim 12, further including the step of:
positioning a cannula adjacent a region of the intervertebral disc to be treated; and
passing the flexible guidable region of the probe through a lumen defined in the cannula.

16. The method according to claim 15, wherein the cannula includes an arcuate portion adjacent a distal end thereof and wherein, during the step of introducing the flexible guidable region of the probe, the arcuate cannula portion guides the flexible guidable region of the probe adjacent to the region to be treated.

17. The method according to claim 16, further comprising the step of:
articulating the arcuate portion of the cannula to a desired orientation within the intervertebral disc.

18. The method according to claim 17, further comprising the step of:
monitoring impedance of tissue to detect variations in tissue-type to thereby facilitate positioning of the flexible guidable region of the probe.

19. The method according to claim 18, further comprising the steps of:
increasing an amplitude of thermal or electromagnetic energy supplied to the probe until indications of effect on the intervertebral disc are obtained; and
noting the amplitude at which the indications of effect of the intervertebral disc are obtained.

20. The method according to claim 19, wherein when the indications of effect of the intervertebral disc are obtained for amplitudes above about 1.25 volts, the method includes the step of applying thermal energy at about 70° C.
